(19) United States
(12) Patent Application Publication
(10) Pub. No.: US 2007/0032558 A1
(43) Pub. Date: Feb. 8, 2007

(54) Method of treating herpes viral infections using a halogenated hydrocarbon composition

(76) Inventors: Laran J. Lerner, Westland, MI (US); Kevin S. Lerner, Westland, MI (US); Jason Sean Lerner, Westland, MI (US); Trevor Matthew Lerner, Westland, MI (US)

Correspondence Address: BROOKS KUSHMAN P.C., 1000 TOWN CENTER, TWENTY-SECOND FLOOR, SOUTHFIELD, MI, 48075, US

(21) Appl. No.: 11/543,512

(22) Filed: Oct. 5, 2006

Related U.S. Application Data
(63) Continuation-in-part of application No. 11/078,222, filed on Mar. 11, 2005.
(63) Continuation-in-part of application No. PCT/US05/08118, filed on Mar. 11, 2005.
(60) Provisional application No. 60/552,585, filed on Mar. 11, 2004.

(51) Int. Cl. A61K 31/522; A61K 31/02; A61K 31/513

(52) U.S. Cl. 514/743; 514/263.31; 514/269

(57) ABSTRACT

A method to reduce the incidence and time to healing of paraesthesia and skin lesions associated with herpes virus infections. In one form, the method of treatment involves topical application with an halogenated hydrocarbon alone, preferably ethyl chloride, which as a skin refrigerant, freezes the virus infected skin cells or subcutaneous skin cells. The desired result is a slowing or halting of the DNA replication cycle of the virus. Alternatively, the composition may be combined with an antiviral agent and compound to reduce pain and itching. The step of freezing the virus at the first signs of a skin lesion reduces the time to healing by allowing the body's own immune system a chance to attack the viral antigens thereby reducing the incidence and severity of skin lesions associated with herpes virus infections.

### CROSS-REFERENCE TO RELATED APPLICATIONS

This application is a continuation-in-part of U.S. Ser. No. 11/078,222 filed Mar. 11, 2005 and PCT Application No. PCT/US2005/008118 filed in the United States on Mar. 11, 2005, which claims the benefit of U.S. provisional application Ser. No. 60/552,585 filed Mar. 11, 2004, now abandoned. Each application is incorporated herein by reference.

### BACKGROUND OF THE INVENTION

1. Field of the Invention

The invention relates generally to a method for reducing the time to healing associated with viral infections and reducing the severity of skin lesions in a host such as a mammal, and more particularly to a process for treating the prodromal stage and skin lesions associated with herpes group viral infections utilizing a topical treatment composition containing a designated organic active ingredient (DOIA). Most particularly, the present invention relates to topical, intradermal and subdermal application of halogenated hydrocarbons (HH), also known as vapocoolants or skin refrigerants from the alkyl halide subgroup, to treat the signs and symptoms of active herpes virus skin lesions in humans.

2. Background Art

Skin lesions and blisters associated with herpes group viruses are well known in clinical medicine. Herpes group viruses are DNA viruses known to infect a wide variety of organisms. Various trigger factors can induce reactivation of the virus after an initial infection, and outbreaks of active lesions can occur throughout the life of an once infected individual. These outbreaks of skin lesions last from 7 to 14 days for herpes simplex type I, type II and chickenpox (varicella-zoster) and for several months for shingles outbreaks. They are often made more painful by contact with clothing.

One particular feature of herpes virus biology relates to the virus' tendency to remain in ganglia of an infected host in a latent state after an initial infection. This latency period is understood to allow the virus to escape total destruction by the host immune system, and may contribute to resistance to various drug therapies.

There are four herpes subgroup viruses known to cause infection and disease in humans. The four subgroups are: (1) herpes simplex virus type I and type II; (2) varicella-zoster virus; (3) cytomegalovirus; and (4) Epstein-Barr viruses. Herpes virus infection by cytomeglovirus and Epstein-Barr viruses do not generally present with skin lesions. It would be desirable to improve treatment protocols of skin lesions associated with herpes simplex type I, herpes simplex type II and varicella-zoster virus infections. Clinical conditions most commonly associated with herpes simplex type I include the familiar cold sores and fever blisters appearing around the mouth and other mucous membranes of infected individuals, known clinically as herpes labialis. Herpes simplex type II is more commonly associated with lesions of the genital areas, and infections are known clinically as herpes progenitalis. Varicella-zoster is associated with the familiar diseases, chickenpox and shingles in humans.

The herpes simplex virus (HSV) is a large (150-200 nm) DNA virus which consists of approximately 152,000 base pairs of double stranded DNA encapsulated in a proteinaceous capsid. The capsid is surrounded by a less well defined structure known as the tegument. The virus is contained in a host cell-derived lipid bilayer, which is studded with virus specific glycoproteins and integral membrane proteins. The HSV is a rapidly replicating DNA virus and the most effective treatments act as replication inhibitors.

Recurrent herpes genitalis are estimated to affect more than 45 million people in the USA alone. (Fleming et al., NEJM, Oct. 97). The incidence is increasing, leading to a massive reservoir for transmission of infection to sexual partners and newborn infants. Once infected, more than 95% of patients who have proven primary herpes genitalis have had at least one recurrence with an average of 5-6 recurrences per year. Approximately 25% of people will have recurrences at monthly intervals accompanied by extremely troublesome physical discomfort and (often) psychological upset. Current therapy for genital herpes revolves around psychological support, education, barrier methods of contraception and treatment on a regular basis with expensive antiviral drugs such as Acyclovir, Pencyclovir and Famcyclovir. These are not well tolerated by all sufferers and in some cases are ineffective.

Herpes simplex infection may be spread from mother to fetus. This usually occurs during parturition but in rare cases can occur in utero. The rate of transmission depends on whether the mother is suffering primary herpes, where the transmission rate is 50% or recurrent herpes where the transmission rate is 8%. If any lesions are visible during the onset of labor, this would be an indication for undertaking a caesarean section. About 50-60% of infants with neonatal herpes simplex virus infection are born to mothers with no history of genital herpes infection. Neonatal herpes simplex infection is very serious. More than 70% of cases present infections localized to the eyes, skin or mouth within 3-30 days of birth. Unfortunately, more than three quarters of those infected will progress to disseminated or CNS involvement which carries a very high morbidity and mortality rate. Treatment of the pregnant mother with oral antivirals is only used when the benefit of treatment of the mother outweighs the risk to the fetus.

Shingles, or herpes zoster, is a common viral infection of the nerves, which results in a painful rash of small blisters on any area of the skin anywhere on the body. Shingles is caused by the same virus that causes chickenpox. Once a person has had chickenpox, the virus lies dormant in the nervous system for many years. Advancing age and/or a lowered immune system seem to be the main causes of shingles. There is no known cure for shingles and treatment focuses on pain relief and treatment of skin lesions with calamine lotion and cold compresses. According to the Centers for Disease Control (CDC), shingles affects an estimated 600,000 to one million people each year.

The herpes viruses also have a role in other pathology, such as acute encephalitis, ulcerative colitis, and Bell's Palsy and cancer. Herpes group viruses are not only extremely widespread within the general population but the pathological effects can be just as diverse highlighting the urgent need for effective treatments.

Myriad formulations and processes for treating herpes virus infections (HVI) have been developed over the years. Orally administered preparations treat systemic infection and must be timed to coincide with viral outbreaks. Most topical preparations, however, are only known to have achieved marginal success in reducing time to healing of skin lesions and blisters. In general, viral infections have historically proven difficult to treat, and herpes virus infections especially so. This is believed to result, at least in part, from the virus' insulation from a host's immune system during the latency period inside the host's nerves.

Cold therapy, also known as cryotherapy, with liquid nitrogen is well known for the treatment of warts. Most warts are associated with Human Papillomavirus (HPV) infections. The process of treatment is to repeatedly freeze the warts with liquid nitrogen, at one to three week intervals to destroy the infected skin cells. After 3-4 months of regular treatment, success is on the order of 70%. However, excessive freezing will destroy the wart and may leave a permanent white mark or scar.

Cold therapy in the field of pre-anesthesia utilizes halogenated hydrocarbons for localized numbness of the skin surface. Doctors, dentists, veterinarians and anaesthesiologists use localized spray applications of known skin refrigerants to temporarily freeze the skin to allow painless insertion of a needle or catheter.

The use of halogenated hydrocarbon treatments that may provide temporary relief of herpes virus skin lesions include ethyl chloride (chloroethane) and fluori-methane, which are chemicals that cool the blood vessels in the skin. These cold therapy treatments have been tried with mixed results in the United States and abroad, but were largely abandoned in the 1980's with FDA approval in 1982 of one of the first HSV antiviral's—Zovirax® (acyclovir). Originally manufactured as topical, oral and injectable branded drug products Zovirax® is now available as the generic Acyclovir. Acyclovir, along with famcyclovir, valacyclovir and pencyclovir are the leading antiviral agents for topical and oral treatment of herpes simplex type II infections.

The Center for Disease Control (CDC) states that between 1970 and 1990 genital herpes infection with Herpes simplex type II have increased 30%. Although the antiviral agents have proven successful in most cases, daily dosage requirements of 500 mg to 1000 mg, and access to prescription drugs within the optimal treatment time frame have made compliance difficult for patients. Even with optimal treatment, the HSV infections are not eliminated and outbreaks of skin lesions still occur, although less frequently.

The body's immune system responds to the herpes viral outbreak approximately 1-2 days prior to the appearance of skin lesions, and begins making antibodies against the viral antigens—a process which can take approximately 2-6 days. This response is usually shortened after the first several outbreaks as the body more quickly recognizes the viral antigens. It has been postulated that a cold therapy method of treatment allows the body time to produce these antibodies which can then attack and destroy the antigen, which is the rapidly-replicating herpes virus, and this will limit the virus's ability to cause further tissue destruction. Similarly, prophylactic cold therapy treatment may be undertaken when the patient experiences other potential trigger factors such as a burning, tingling, itching feeling called paraesthesia.

Although there is now a vaccine Varivax® approved by the U.S. FDA in 1995 to prevent or reduce the pathology of chickenpox, there are no studies confirming treatment with this vaccine is beneficial in patients who experience outbreaks of shingles. Shingles is still treated with pain relievers, bed rest and cold compresses.

### SUMMARY OF THE INVENTION

The invention includes a method for treating skin lesions associated with herpes viral infections in a host. The method reduces the duration and severity of the lesions associated therewith. One objective of the method of treatment is to shorten the course of the herpes viral lesions and thereby lessen transmission and spread to others. An additional benefit and effect on society are the decreased healing time that lessens days missed from work and/or school.

A patient presents to a health care provider with signs and symptoms of a herpes viral infection which may include skin lesions or pain, itching, burning and tingling, also known as paraesthesia. The patient once diagnosed with a herpes viral infection will be counseled on his/her treatment options.

Accordingly, one aspect of the invention uses a composition for treating skin lesions in a host by applying a therapeutically effective amount of a halogenated hydrocarbon (alkyl halide) compound to the affected tissue in a dosage or with a frequency sufficient to speed healing of the affected tissue. The term “alkyl” means a hydrocarbon having from 1 to 12 carbon atoms unless otherwise specified and includes, for example, methyl and ethyl groups. The alkyl group may be independently substituted by a “halide” from the substituents F, Cl, Br or I. Halogenated hydrocarbons or “alkyl halides” may include organochlorides, methyl chloride and ethyl chloride (alkyl chlorides), alkyl bromides, and alkyl fluorides (such as methyl fluoride). The choice is governed by the halogenated hydrocarbon's ability to act as a vapocoolant or skin refrigerant.

Representatives from this class of compounds are known to evaporate relatively rapidly when applied externally, causing cooling and contracting of blood vessels in the skin and locally alleviating pain. It was heretofore unknown, however, that one time or repeated administration of vapocoolant sprays could speed the healing and prevent recurrence of herpes related skin lesions.

The invention provides a method for treatment utilizing a composition for treatment of skin lesions in a host wherein the application of therapeutically effective amounts of a halogenated hydrocarbon may be combined with other compounds including those that are effective in decreasing the recurrence of the herpes virus and compounds known to relieve the pain and itching associated with skin lesions, in a dosage to speed healing of infected tissue. These compounds may be combined to form treatment compositions or applied separately in any order.

Antiviral agents known to decrease the recurrence of the herpes virus may include acyclovir, famcyclovir, valacyclovir, viral thymidine kinase inhibitor, and any partially effective herpes viral recurrence inhibitors known in the art. Anti-pruritic and analgesic compounds may also be included to relieve the symptoms of pain and itching. Many of these compounds contain hydrocortisone as the active ingredient.

The halogenated hydrocarbon composition can be delivered in several ways, but a preferred storage container is made of firm or malleable sterile material such as a glass or a plastic material that does not interact with the halogenated hydrocarbon dispensed under pressure. A preferred process step of the invention is to configure the composition to evaporate relatively rapidly when applied topically. In our embodiment, this is achieved by formulating the halogenated hydrocarbon or alkyl halide under pressure.

The term “therapeutically effective amount” as used herein refers to an amount of the halogenated hydrocarbon (HH) composition sufficient to decrease the healing time for active herpes lesions and/or to decrease the frequency of reactivation of a herpes virus infection as compared to healing without treatment. The dosage ranges for administration of the halogenated hydrocarbon are those that produce the desired effect.

The invention provides alternate methods for delivery of the treatment compounds by topical, intradermal and subcutaneous routes of administration directly to the skin lesion(s). The treatment should be reapplied as necessary over time until the skin lesions are no longer visible or painful.

Other features and advantages of the present invention will become more apparent to persons having ordinary skill in the art to which the invention pertains from the following description.

### BRIEF DESCRIPTION OF THE DRAWINGS

The treatment steps proposed by FIG. 1 are a guideline and may occur in an order best suited for use by those skilled in the art.

FIG. 1 is a diagram of the treatment steps comprising the invention in the preferred embodiment.

### DETAILED DESCRIPTION OF THE INVENTION

The invention provides for a method of treatment to reduce the time for healing of paraesthesia and skin lesions associated with a herpes virus or any other type of viral skin lesions by use of a halogenated hydrocarbon (alkyl halide) composition.

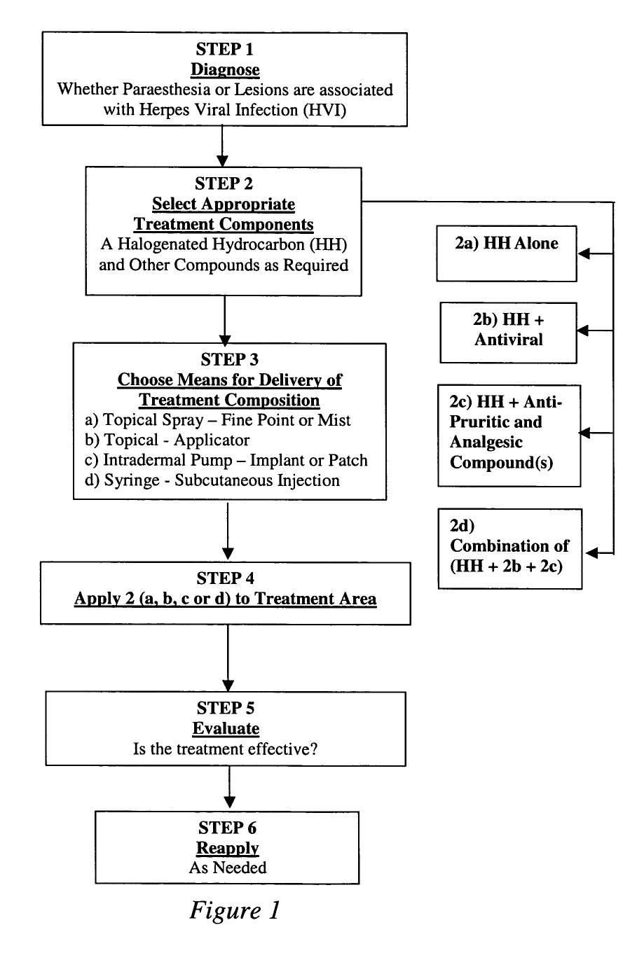

The treatment method is applicable to mammals, most particularly humans, and is contemplated for topical application to lesions resulting from infection by herpes virus, in particular: 1) herpes simplex virus types I and II; and 2) varicella-zoster virus. In one example, the disclosed process is contemplated for use in treating herpes infections affecting the skin and mucous membranes, including but not limited to the mouth, genital, anal and urethral areas as well as peripheral skin surfaces. (FIG. 1, step 1)

In one form, a method of treatment is embodied by the process of storing and applying the halogenated hydrocarbon (HH) (FIG. 1, step 2a) composition in a container that promotes an aerosol mist or spray directed to the skin lesion. (FIG. 1, step 3) The application dosage could include a number of applications separated by a fixed time or as necessary when the lesion is again painful. (FIG. 1, steps 4, 5, 6)

The inventive protocol is thought to be a physical freezing of the virus' DNA, which may occur at temperatures below 8 degrees celsius. The halogenated hydrocarbon (HH) (FIG. 1, step 2a) composition should be a “therapeutically effective amount” so as to provide a temporary ‘frostnip’ or a localized numbing effect between 3 to 10 degrees celsius which should decrease the skin's sensation of pain and not cause further edema of the skin. While not being bound to any specific theory, the time it takes the viral DNA to freeze and restart replication, upon thawing, is thought to give the body's own immune system a chance to attack the viral antigens.

During the treatment process, it may also be desirable to continue application (FIG. 1, step 4) of the treatment composition (FIG. 1, step 2) to the affected area after the herpes lesions heal over to prevent self-reinfection from leaking lesions.

One topically applied composition contains a halogenated hydrocarbon (HH) compound such as organo- or alkyl halides, preferably organochlorides. (FIG. 1, step 2a) Halogenated hydrocarbons are presently known and used for example, as vapocoolant or skin refrigerant materials for topical pain treatment. Exemplary compounds include ethyl chloride (chloroethane) and fluorimethane (methyl fluoride). Less preferred compounds include more complex halogenated hydrocarbons such as chloro-di-fluori-methane.

Chloroethane sprays sold under the trade name Fluori-Methane® (diclorodifluoromethane 15% and trichloromonofluoromethane 85%) are available commercially from the Gebauer Company of Cleveland, Ohio. Other vapocoolant sprays are commercially available, such as those sold under the trade name Frigiderm® (dichlorotetrafluoroethane) from Delasco Corp. of Council Bluffs, Iowa.

The class of suitable compounds include alkyl chlorides, alkyl bromides, and alkyl fluorides. Particularly preferred alkyl chlorides include methyl chloride and ethyl chloride. Certain halogenated hydrocarbons are known to be toxic when ingested or absorbed through the skin. Where the relatively more toxic halogenated hydrocarbons are used, they may be diluted in a liquid carrier to a concentration that is harmless. If desired, preservatives and other additives may also be present in the treatment composition such as, for example, carriers, other analgesics, antimicrobials, anti-oxidants, chelating agents, inert gases, and the like. (FIG. 1, step 2)

A person of ordinary skill in the art, given the teachings of the present specifications, may readily determine suitable dosage ranges, by varying concentrations of halogenated hydrocarbon in the mixture applied and/or by varying the frequency of application (FIG. 1, step 4). The dosage can be adjusted by the individual physician in the event of any contraindications. In any event, the effectiveness of treatment may be determined by monitoring the recurrence of lesions (FIG. 1, step 5) by examination methods well known to those in the field. The means of delivery of the composition may also be varied by use of either a fine point spray to treat a specific lesion or a mist spray to cover several lesions in a given area of the body (FIG. 1, step 3a).

The halogenated hydrocarbon may also be combined with compounds known to be effective in reduction in pain and itching (e.g. hydrocortisone) (FIG. 1, step 2c) and compounds known to be effective in decreasing the recurrence of herpes viruses, e.g., acyclovir, famcyclovir, valacyclovir, viral thymidine kinase inhibitors, and other partially effective herpes recurrence inhibitors (FIG. 1, step 2b).

Treatment compositions contemplated describe: a) the halogenated hydrocarbon alone (FIG. 1, step 2a), b) as a combination containing the halogenated hydrocarbon, and an antiviral agent (FIG. 1, step 2b), c) the halogenated hydrocarbon and a compound to decrease pain and itching (FIG. 1, step 2c) or d) the halogenated hydrocarbon, an antiviral agent and a compound to decrease pain and itching (FIG. 1, step 2d). Alternatively, the treatment method contemplated may be applied sequentially as separate compounds.

The invention includes a method of treatment wherein cold therapy is applied topically, intradermally or subcutaneously to skin cells infected by a virus (FIG. 1, steps 3a, 3b, 3c and 3d). At present, there is no method to apply cold therapy parenterally for a systemic virus, which circulates throughout the body.

In one preferred embodiment, the halogenated hydrocarbon composition (FIG. 1, step 2a) is applied directly in a spray form (FIG. 1, step 3a) to active herpes lesions one or more times per day, at a frequency and in a concentration sufficient to speed healing. As an alternative to spraying the halogenated hydrocarbon (FIG. 1, step 2a), it may be directly applied, for example, with a spray- or liquid-saturated applicator (FIG. 1, step 3b). An applicator may be a cotton ball, a swab or similar means of topical application. Another alternative contemplated is intradermal infusion of the treatment composition under pressure via a pump, patch (FIG. 1, step 3c) or injection device (FIG. 1, step 3d).

An additional benefit of cold therapy treatment with the treatment compositions results from delivery in a fine point spray (FIG. 1, step 3a). Herpes lesions have a pre-vesicle stage, an intermediate fluid filled vesicle stage and a post-vesicle “crusted” ulcer stage. The force of delivery of a fine point spray as opposed to a mist spray of the present composition to the fluid-filled vesicle stage has the potential to break the skin of the fluid filled vesicle causing the fluid to freeze or cool and by intradermal penetration slow viral DNA replication.

Alternative embodiments are contemplated, as described above wherein the halogenated hydrocarbon (HH) composition is delivered intradermally (FIG. 1, step 3c) rather than topically (FIG. 1, steps 3a and 3b), for example, in the form of a slow-release subcutaneous implant or an epidermal patch (FIG. 1, step 3c). The compound may alternatively be delivered by hypodermic injection (FIG. 1, step 3d) with a suitable carrier fluid and means for delivery of the halogenated hydrocarbon under pressure, into and around the affected tissue.

The present description is for illustrative purposes only, and should not be construed to limit the scope of the present invention in any way. Thus those skilled in the art will appreciate that various departures might be made from the presently disclosed embodiments without departing from the full and fair scope of the present invention.

What is claimed is:

1. A method of treating herpes viral infections (HVI), the method comprising the steps of:
(1) diagnosing whether a patient's condition is associated with a herpes viral infection;
(2) selecting a treatment composition from a group consisting of: a) a halogenated hydrocarbon, b) a halogenated hydrocarbon plus an antiviral agent, c) a halogenated hydrocarbon plus an anti-pruritic and an analgesic, d) a halogenated hydrocarbon plus an antiviral agent plus an anti-pruritic and an analgesic, and (e) mixtures of compositions (a-d).
(3) choosing a means for delivery of a treatment composition, a delivery choice selected from the group consisting of a topical spray, a fine point spray, a mist spray, direct topical application, an intradermal pump, an implant, a patch, subcutaneous injection and combinations thereof;
(4) applying a treatment composition to an area of skin to be treated through a chosen means for delivery;
(5) evaluating effectiveness of a treatment composition applied through a chosen means for delivery; and
(6) reapplying a treatment composition as needed.

2. The method of claim 1, wherein the antiviral agent in step 2(b) is selected from the group consisting of acyclovir, famcyclovir, valacyclovir, viral thymidine kinase inhibitors, and other herpes viral recurrence inhibitors that are effective in decreasing recurrences of herpes virus infections.

3. The method of claim 1, wherein step 4 comprises delivering the halogenated hydrocarbon, the antiviral agent and the anti-pruritic and analgesic compounds in any order.

4. The method of claim 1, wherein step 4 comprises application of the halogenated hydrocarbon followed by application of the antiviral agent, the anti-pruritic and analgesic agent.

5. The method of claim 1, wherein step 4 comprises application of the halogenated hydrocarbon preceded by application of the antiviral agent, the anti-pruitic and analgesic agent.

6. The method of claim 1, wherein step 2(c) comprises the step of selecting the treatment composition such that the treatment composition is effective in decreasing pain and itching associated with skin lesions.

7. The method of claim 6, wherein the topical application of halogenated hydrocarbon is followed by application of the anti-pruritic and the analgesic.

8. The method of claim 6, wherein the topical application of halogenated hydrocarbon is preceded by application of the an anti-pruritic and an analgesic.

9. The method of claim 1, wherein step 3 comprises delivering the halogenated hydrocarbon composition by dispersing the composition as a fine point spray for topical application.

10. The method of claim 1, wherein step 3 comprises delivering the halogenated hydrocarbon composition by dispersing the composition as a mist spray for topical application.

11. The method of claim 1, wherein step 3 comprises delivering the halogenated hydrocarbon composition utilizing an applicator.

12. The method of claim 1, wherein step 3 comprises delivering the halogenated hydrocarbon composition utilizing a pump connected to a skin implant for intradermal administration.

13. The method of claim 1, wherein step 3 comprises delivering the halogenated hydrocarbon composition utilizing a pump connected to a patch for topical application.

14. The method of claim 1, wherein step 3 comprises delivering the halogenated hydrocarbon composition utilizing a syringe for subcutaneous administration.

15. A method of treating viral lesions by applying a treatment composition to a human, wherein the method comprises:
(1) applying to the human a form of treatment comprising a treatment composition selected from the group consisting of:
a) a halogonated hydrocarbon;
b) a halogonated hydrocarbon and an antiviral agent;
c) a halogonated hydrocarbon plus an anti-pruritic and an analgesic;
d) a halogonated hydrocarbon plus an antiviral agent plus an anti-pruritic and an analgesic, and
e) mixtures thereof.
(2) a treatment composition which is an antiviral therapy, wherein the delivery of treatment comprises a means selected from the group consisting of a topical spray, a fine point spray, a mist spray, direct topical application, an intradermal pump, an implant, a patch, subcutaneous injection and combinations thereof; and
(3) administering a selected treatment composition over a selected period of time in which a therapeutically responsive dose is utilized to promote healing of skin lesions.
